(19) United States
(12) Patent Application Publication
(10) Pub. No.: US 2019/0038791 A1
(43) Pub. Date: Feb. 7, 2019

(54) Medical Device Inspection and Sterilization

(71) Applicant: Sanovas Intellectual Property, LLC, Reno, NV (US)

(72) Inventors: Lawrence J. Gerrans, San Anselmo, CA (US); Kaan Erol, San Francisco, CA (US); Oscar Pantoja, San Lorenzo, CA (US)

(21) Appl. No.: 16/053,336

(22) Filed: Aug. 2, 2018

Related U.S. Application Data
(60) Provisional application No. 62/540,312, filed on Aug. 2, 2017.

(51) Int. Cl. A61L 2/22; A61B 90/70; A61L 2/10; A61L 2/18; A61L 2/20; B08B 3/08; B08B 9/043; B08B 1/00; B08B 7/00; B08B 7/04

(52) U.S. Cl. CPC A61L 2/22; G06T 7/0004; A61L 2/10; A61L 2/18; A61L 2/206; B08B 3/08; B08B 9/0436; B08B 1/002; B08B 7/0057; B08B 7/04; A61B 2090/702; A61B 2090/701; A61L 2202/11; A61L 2202/14; A61L 2202/15; A61L 2202/17; A61L 2202/24; A61B 90/70

(57) ABSTRACT

A method of ensuring the structural integrity and decontamination of a medical device includes inserting a distal end of a borescope into a lumen of the medical device, where the distal end of the borescope has an imaging portion and at least one decontamination portion, typically a UV light and a fluid sterilant or bactericide. The method includes determining both whether the lumen wall has been damaged, and whether contaminants reside on the wall, by visually inspecting the wall with the imaging portion of the borescope, and then decontaminating the wall with the decontaminating portion of the borescope. Device processing data digitally associated with the medical device is retrieved with a unique device identifier, and additional device processing data resulting from the current inspection and sterilization process is added for use by subsequent device handlers, typically by adding it to a distributed ledger, such as a blockchain.

### FIELD OF THE INVENTION

The present invention relates to the inspection and sterilization of medical instruments. More specifically, the invention relates to employing a borescope inserted into a lumen of a medical instrument that both visualizes and sterilizes the interior of the lumen to ensure the structural integrity and decontamination thereof, and documenting this to track the history of same during the life of the instrument.

### BACKGROUND OF THE INVENTION

Many medical instruments, such as endoscopes, catheters, cannulas, and guidewire-driven instrumentation contain one or more inner channels or lumens through which biological material, fluids, pharmaceuticals, working devices, and other substances travel. Consequently, these small diameter lumens usually become soiled. If this is not sufficiently addressed, subsequent use of these devices can lead to serious infections or adverse reactions in subsequent patients. Therefore, it is important to adequately identify any such contamination, and clean and decontaminate these lumens after each surgery or clinical use.

Additionally, in view of the various types of devices and materials often used in these lumens, the medical device will sometimes incur internal damage from physical force or wear, or from corrosion. It is important to identify any such damage in the device lumen for several reasons. First, it is important to know if the structural integrity of the device has been compromised so that it can be repaired, or its use discontinued, before there is a dangerous device failure during a medical procedure. In some cases, the wall of the device can even be punctured from within the lumen. Moreover, even if the wall of the lumen only gets scratched, this creates a breading ground for infectious bacteria, further exacerbating any contamination. Thus, the identification of any such damage is critical.

It is also important to know the inspection and sterilization history of the medical devices prior to using that device. Many devices that have been processed and made available for the next use are actually still contaminated. Accordingly, it is vitally important that the inspection and sterilization procedures be documented and made available for review by subsequent medical practitioners and supporting personnel so that they can be sure it has been properly processed before it is used in the next procedure.

What is desired, therefor, is a system for determining whether the structural integrity of the walls defining lumens in medical devices has been compromised and the medical device is in need of repair. What is further desired is a system for determining whether the walls of medical device lumens are contaminated with biological material or other residue and in need of cleaning. What is also desired is such a system that can sterilize the walls of medical device lumens found to be contaminated. What if still further desired is a system that keeps track of any such inspection, repair, cleaning, and sterilization for future review.

### SUMMARY OF THE INVENTION

Accordingly, it is an object of the present invention to provide a system that enables visualization of the lumens of medical devices to assess damage or contamination of the lumen walls.

It is also an object of the present invention to provide a system that facilitates sterilization of the lumens of medical devices.

It is a further object of the present invention to provide such a system that enables the documentation of inspection, repair, cleaning, and/or sterilization of the medical devices.

In order to overcome the deficiencies of the prior art and to achieve at least some of the objects and advantages listed, the invention comprises a method of ensuring the structural integrity and decontamination of a medical device, including inserting a distal end of a borescope into a lumen of a medical device, the distal end of the borescope having an imaging portion and at least one decontamination portion, determining whether the wall of the lumen has been damaged by visually inspecting the wall of the lumen with the imaging portion of the borescope, determining whether contaminants reside on the wall of the lumen by visually inspecting the wall of the lumen with the imaging portion of the borescope, and decontaminating the wall of the lumen with the decontaminating portion of the borescope.

In certain advantageous embodiments, the at least one decontaminating portion comprises an ultraviolet light source, and the step of decontaminating the wall of the lumen comprises sterilizing the wall with ultraviolet light from the ultraviolet light source.

In some embodiments, the at least one decontaminating portion comprises a fluid delivery device, and the step of decontaminating the wall of the lumen comprises delivering a fluid decontaminant to the wall via the fluid delivery device. In some cases, the step of decontaminating the wall of the lumen comprises sterilizing the wall by delivering a fluid sterilant thereto via the fluid delivery device. In other cases, the step of decontaminating the wall of the lumen comprises delivering a fluid bactericide thereto via the fluid delivery device.

In certain embodiments, the fluid sterilant comprises peracetic acid. In other cases, the fluid sterilant comprises hypochlorous acid. In still other cases, the fluid sterilant comprises ethylene oxide.

In some embodiments, the fluid delivery device comprises a nebulizer for delivering an atomized liquid to the wall of the lumen.

In certain advantageous embodiments, the method further includes retrieving device processing data digitally associated with a unique device identifier for the medical device, and adding to the device processing data additional device processing data resulting from the steps of determining whether the wall of the lumen has been damaged, determining whether contaminants reside on the wall of the lumen, and decontaminating the wall of the lumen. In some of these embodiments, the step of adding to the device processing data comprises adding the additional device processing data to a distributed ledger. In some embodiments, the step of adding the additional device processing data comprises adding the information to a blockchain.

In certain embodiments, the step of retrieving device processing data digitally associated with a unique device identifier comprises scanning the unique device identifier with the imaging portion of the borescope.

In some embodiments, the method further includes the following steps, after the step of determining whether contaminants reside on the wall of the lumen and before the step of decontaminating the wall of the lumen: withdrawing the distal end of the borescope from the lumen of the medical device, inserting a catheter having a balloon with a mesh thereon into the lumen, cleaning the lumen with the balloon, withdrawing the catheter from the lumen, and reinserting the borescope into the lumen. In other embodiments, the method includes the following steps, after the step of determining whether contaminants reside on the wall of the lumen and before the step of decontaminating the wall of the lumen: withdrawing the distal end of the borescope from the lumen of the medical device, inserting a brush into the lumen, cleaning the lumen with the brush, withdrawing the brush from the lumen, and reinserting the borescope into the lumen.

In some embodiments, the method includes the step of sending the medical device for repair if the step of determining whether the wall of the lumen has been damaged determines that the wall has been damaged.

In certain embodiments, the method also includes the steps of capturing an image of the wall of the lumen while visually inspecting the wall with the imaging portion of the borescope, and storing the image. In some embodiments the method also includes the steps of recording a video of the wall of the lumen while visually inspecting the wall with the imaging portion of the borescope, and storing the video.

In certain advantageous embodiments, the imaging portion comprises a CMOS sensor.

### BRIEF DESCRIPTION OF THE DRAWINGS

FIG. 1 is a schematic view of a system according to the invention.

FIG. 2 is a schematic view of the proximal end of the borescope used in the system shown in FIG. 1.

FIG. 3 is a schematic view of the distal end of the borescope used in the system shown in FIG. 1.

FIG. 4 is a schematic view of visualizing the lumen of a medical device in accordance with the invention of FIG. 1.

FIG. 5 is a schematic view of cleaning the lumen of a medical device with a balloon catheter.

FIG. 6 is a schematic view of cleaning the lumen of a medical device with a brush.

FIG. 7 is a schematic view of sterilizing the lumen of a medical device using ultraviolet light in accordance with the invention of FIG. 1.

FIG. 8 is a schematic view sterilizing the lumen of a medical device using a nebulized sterilant in accordance with the invention of FIG. 1.

FIG. 9 is a flow chart illustrating the operation of part of the system of FIG. 1.

FIG. 10 is a flow chart illustrating the operation of part of the system of FIG. 1.

### DETAILED DESCRIPTION OF THE INVENTION

The following detailed description illustrates the technology by way of example, not by way of limitation, of the principles of the invention. This description will enable one skilled in the art to make and use the technology, and describes several embodiments, adaptations, variations, alternatives and uses of the invention, including what is presently believed to be the best mode of carrying out the invention. One skilled in the art will recognize alternative variations and arrangements, and the present technology is not limited to those embodiments described hereafter.

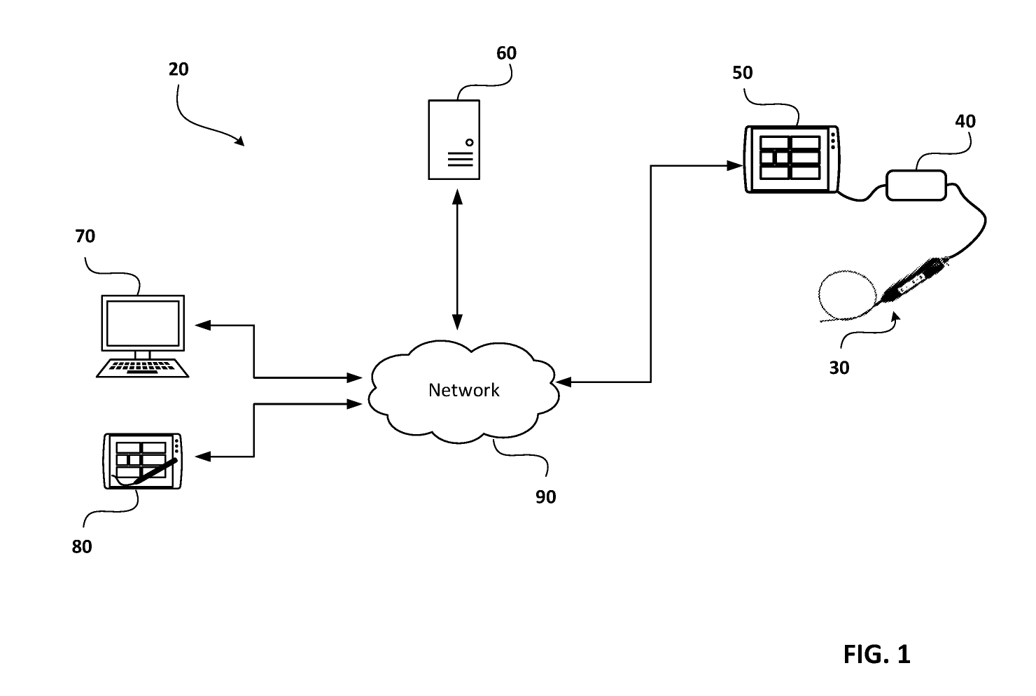

FIG. 1 illustrates one exemplary embodiment of a system (20) in accordance with the invention. A borescope (or imaging catheter) (30) is in communication with a processor (40) containing hardware and/or software for processing imaging data received from the borescope (30). The processor (40) is connected to a display device (50) for receiving and displaying the processed imaging data to a medical practitioner. While the processor (40) is shown separately from the display device (50), it should be noted that processor (40) can also be part of the display device (50). The proximal end of the catheter can be connected directly to a portable or stationary display device for displaying the images from the camera, such as, for example, a tablet, or it can be connected to a more substantial processing system, such as a personal computer or larger network, which can facilitate the device documentation and tracking procedures further described below.

The processor (40) and/or display device (50) are in communication with a server (60) over a network (90), such that data can be communicated to the server (60), from which other devices (70), (80) can access the data. While the network (90) will often be the Internet, the network could also be a local intranet, such as a hospital network.

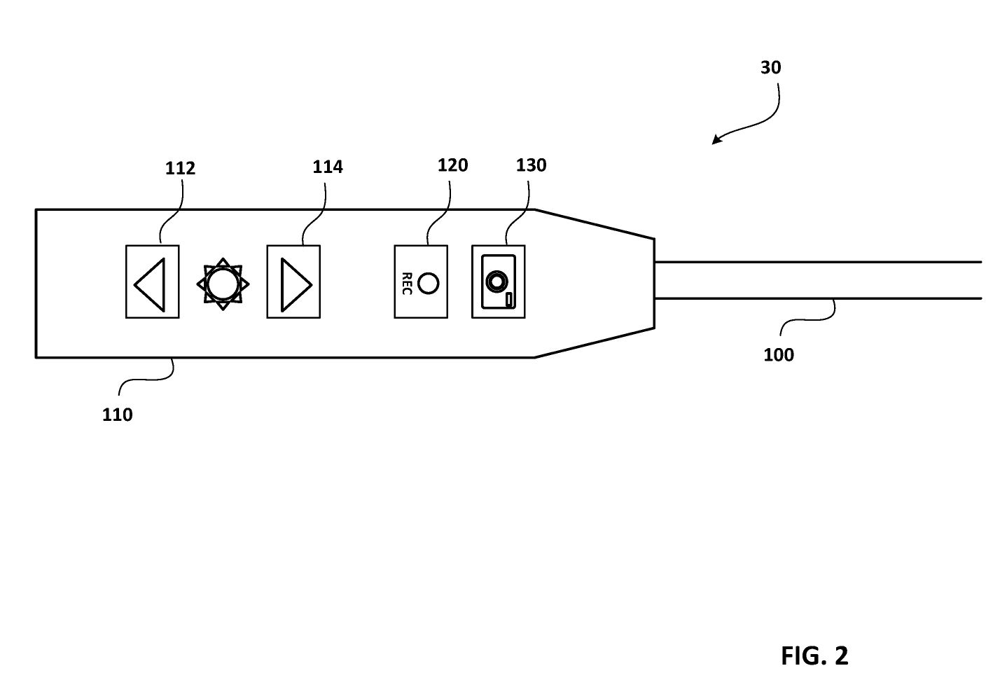

Referring to FIG. 2, the borescope (30) includes an elongated body portion (100) and a handle portion (110). In addition to brightness controls (112)-(114), the handle (110) includes an image capture button (120) that, when pressed, sends a command to the processor (40) and/or display device (50) to capture a still image of the current view of the borescope (30). Similarly, the handle (110) also includes record button (130) that, when pressed, sends a command to the processor (40) and/or display device (50) to record video of the current view of the borescope (30). These images and videos can be stored in memory located on the processor and/or display device (50), or they can be uploaded via the network to a sever (60).

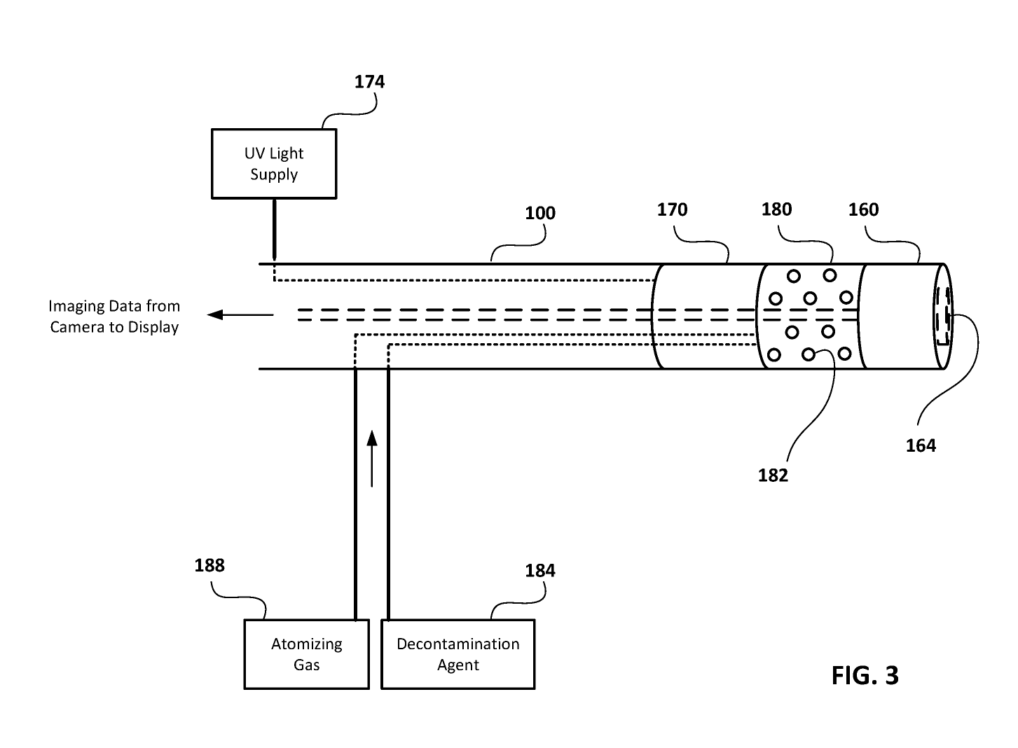

Referring to FIG. 3, the distal end of the borescope (30) is shown. The distal end of the elongated body (100) has a camera (or imager) section (160) for visualizing the lumen of a medical device. The camera section (160) may comprise, for example, a CMOS sensor (164), though other imaging systems, such as CCD sensors or optical fiber, may be employed.

The distal end of the elongated body (100) also has at least one decontamination portion. One such decontamination portion is an ultraviolet light source (170). The light source (170) is supplied with ultraviolet light from a UV light supply (174), such that it can deliver sterilizing UV light. In certain cases, gamma or beta irradiation can be used.

Another such decontamination portion is a fluid delivery device (180) for delivering a decontaminating agent. This fluid can be any appropriate decontaminant, including a sterilant, a bactericide, or a disinfectant. The fluid delivered via the device (180) can be a liquid or gas decontaminant. Some decontaminants that may be used include hypochlorous acid, peracetic acid, and hydrogen peroxide.

For example, in cases where the fluid to be delivered is a liquid, the fluid delivery device can be a nebulizer (180), as illustrated. The nebulizer is in fluid communication with a source of one or more decontaminants (184), as well as a source an atomizing gas (188), such as carbon dioxide. These sources can be external or, for example, can be portable and/or replaceable canisters coupled to, or housed within, a handle for the borescope.

The decontaminating agent (184) is supplied via a conduit through the elongated body (100) to the nebulizer (180). Contemporaneously, the atomizing gas (180) is likewise supplied via a conduit through the elongated body (100) to the nebulizer (180), such that the decontaminating agent (184) is atomized thereby, after which it is emitted through a plurality of apertures (182). In this way, the decontaminant is transmitted to the nebulizer and sprayed on the walls of the lumen.

Alternatively, in lieu of nebulizing a liquid decontaminant such as is described above, or in addition thereto, the fluid delivery device may simply have a delivery port for delivering a gas. One such example is ethylene oxide, which can be used as a sterilant. Other gases, such as nitrogen, or any gas that would starve undesirable bacteria, can be employed.

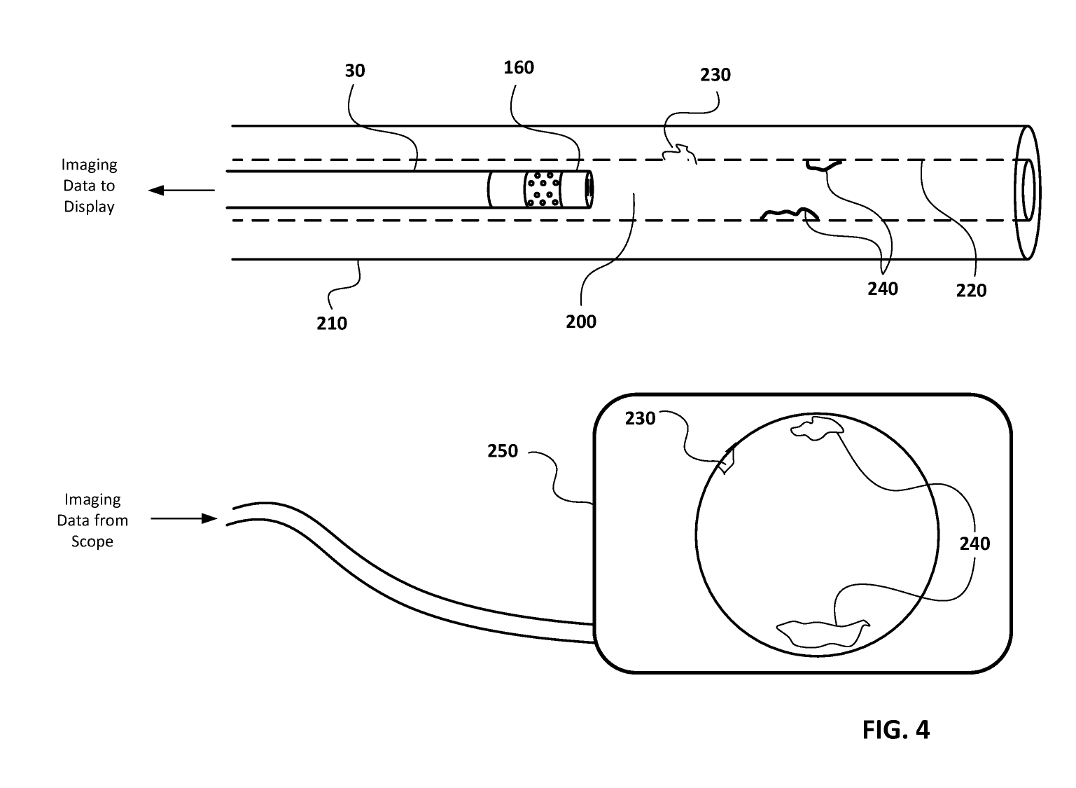

The operation of an exemplary embodiment of the present invention is illustrated in FIGS. 4-8. Referring first to FIG. 4, the distal end of the borescope (30) is inserted into the lumen (200) of a medical device (210). The imaging portion (160) of the borescope (30) is used to visually inspect the wall (220) of the lumen (200) of the medical device to determine if there has been any damage thereto, as may occur, for example, in the case of a working channel of an endoscope, though which a surgical instrument is repeatedly advanced and withdrawn, such that the inner lumen walls become scratched or gouged during operation.

The imaging data obtained by the imaging portion (160) undergoes image processing as noted above and is rendered on a display (250) for the user. By using the imaging portion (160) to perform this inspection of the lumen wall (220) in this way, one can identify any structural infirmities (230) in the wall of the lumen. In the event this visualization reveals there is indeed a damaged portion (230), the medical device (210) is sent for repair prior to any subsequent use in view of the fact that this damage presents a risk of device failure or further increases the likelihood of residual contamination.

The imaging portion (160) of the borescope (30) is also used to visually examine whether the lumen (200) contains any debris, such as microbial or other biological matter, residual pharmaceutical agents, etc., that resides on the wall thereof. When debris (240) is found, the borescope (30) is withdrawn from the lumen (200), which is subsequently cleaned with one of several methods.

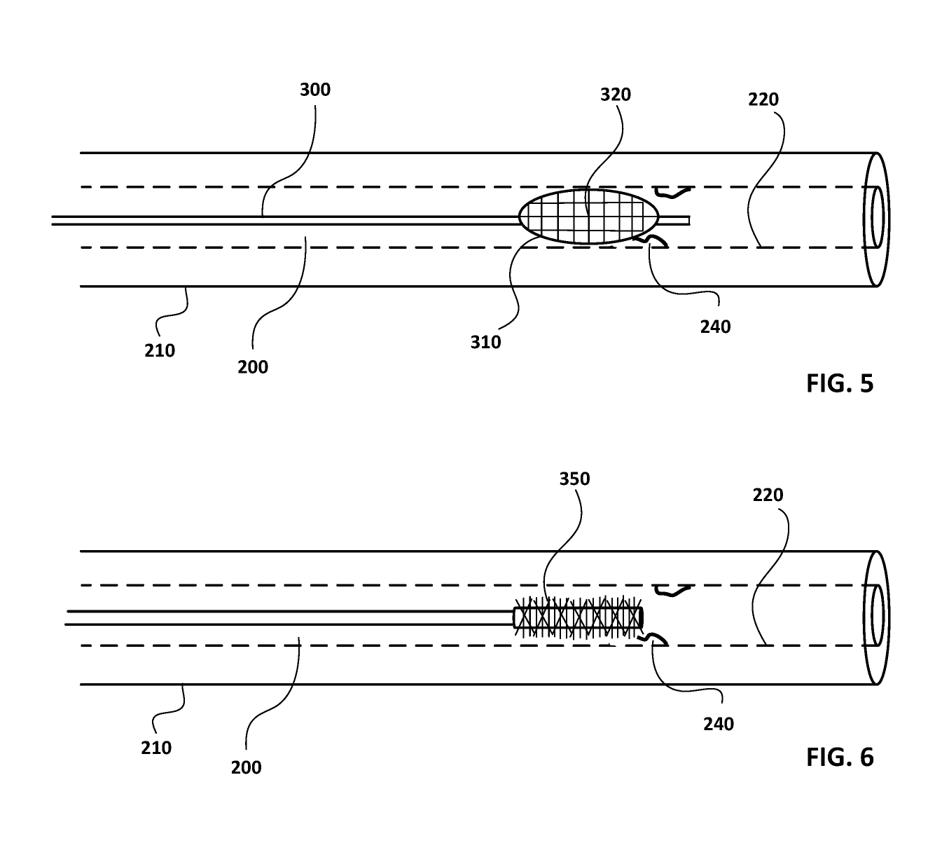

For example, referring to FIG. 5, a balloon catheter (300) can be used for this purpose. The catheter (300) includes a balloon (310) at its distal end. The balloon (310) has a mesh disposed on the outer surface thereof, such that it has certain texture. As described more fully in U.S. Pat. No. 8,226,601 to Gunday et al., the specification of which is incorporated herein in its entirety, this mesh balloon can be advanced past the debris (240), then inflated, and then withdrawn to capture debris and pull it out of the lumen. If necessary, the balloon-covered mesh can be moved back and forth, rotated, or inflated in pulsed fashion, to wear away at a contaminant (240) stuck to the wall (220).

As another example, referring to FIG. 6, a brush (350) can be employed to remove the debris (240). The brush (350) is inserted into the lumen (200), and like the balloon (310), can be moved back and forth or rotated to scrape and capture the debris (240).

In some cases, the imaging portion (160) of the borescope (30) is configured to conduct the visualization in the non-visible spectrum, such as infrared or near-infrared, which can also be used in combination with visualization in the visual spectrum (generally, from a lower limit between 360 and 400 nm and an upper limit between 760 and 830 nm) to further improve the likelihood of identification of undesirable contaminants in the lumen by facilitating the detection of pathologies and contaminants in the non-visible spectrum.

Moreover, additional optical imaging techniques for improving image quality can be used, such as near infrared imaging, confocal microscopy, Bioluminescence imaging, Fluorescence Imaging, Raman imaging, Photoacoustic imaging, Cerenkov luminescence imaging, Cerenkov excited fluorescence imaging, X-ray excited luminescence imaging, and radiopharmaceutical excited fluorescence Imaging.

In certain embodiments, the imaging portion (160) of the borescope (30) is also used as a scanner for a unique device identifier (“UDI”) of the medical device (210), as is explained in further detail below.

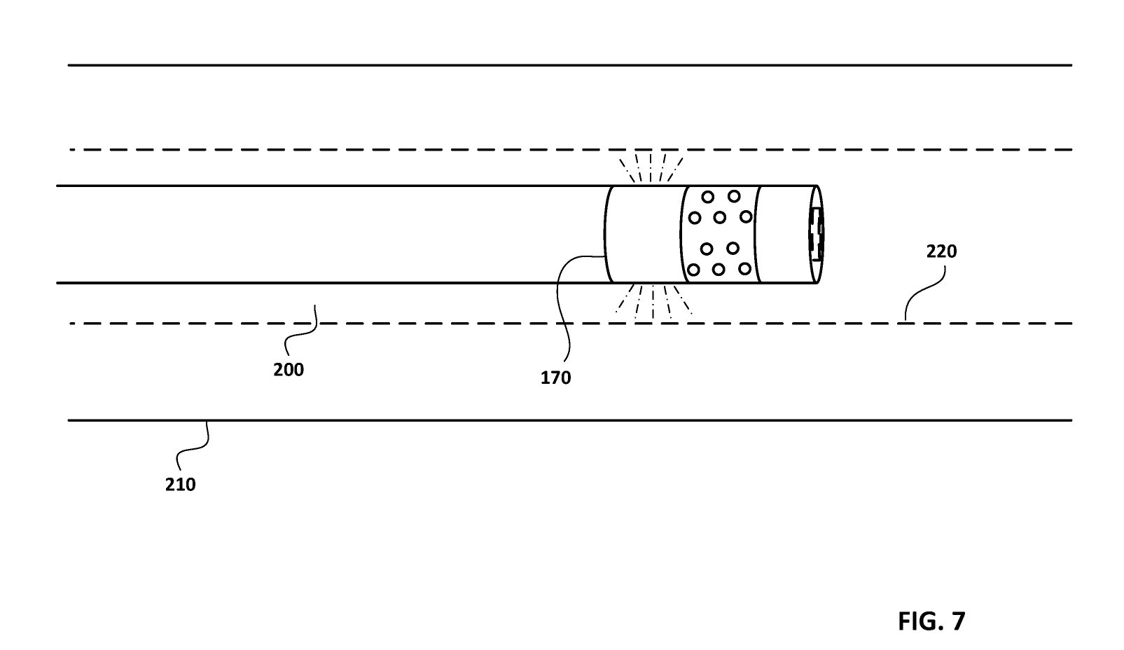

Referring to FIG. 7, the borescope is next used to sterilize the lumen (200). In instances where the borescope (30) had to be removed in order to first remove larger contaminants (debris) as described above, the distal end of the borescope (30) is reinserted. Ultraviolet light is then supplied to the light source (170), which can be activated via a button on the handle (110) or at the supply (174) itself. As a result, the light source (170) radiates this ultraviolet light in three hundred and sixty degrees within the lumen (200), thereby sterilizing the lumen wall (220).

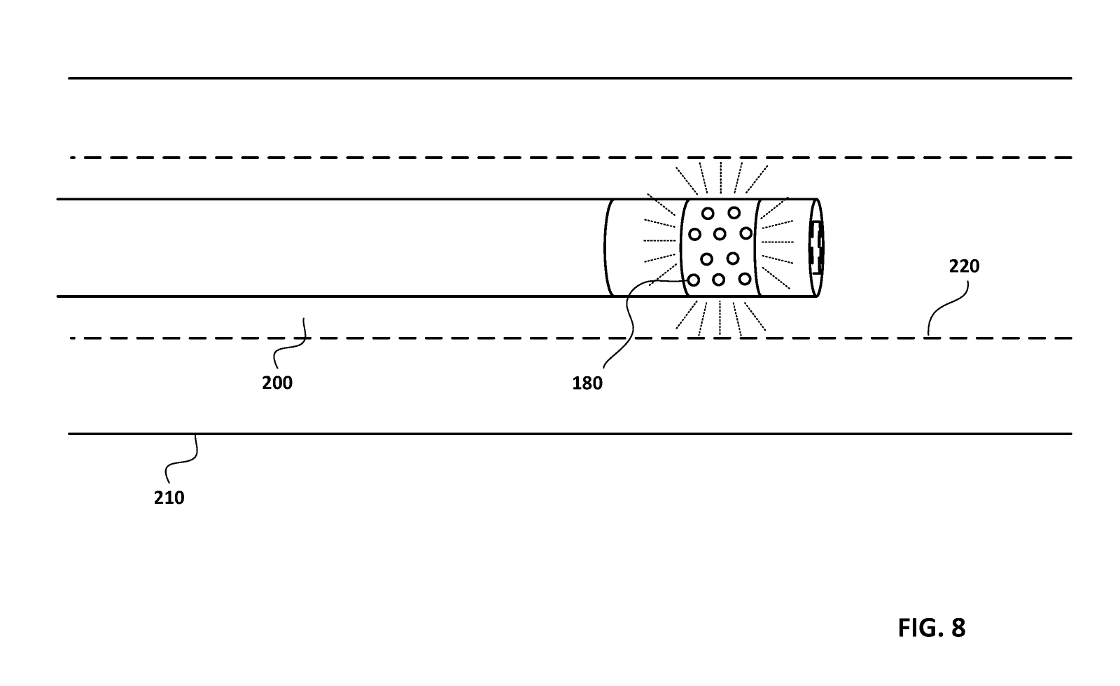

Referring to FIG. 8, the borescope is next used to further ensure full decontamination of the lumen (200) by delivering a fluid sterilant or bactericide targeting some particular bacteria based on the prior use of the medical device (210). In this case, the nebulizer (180) sprays the sterilant or bactericide on the lumen wall (220). It should be noted that, although the system has been described with respect to first sterilizing the lumen (200) with the UV light source (170) and then with the fluid delivery device (180), this is not required, and this order can be reversed.

Additionally, the sterilizing radiation can be used in conjunction with a photosensitizing agent, such as porfimer sodium, to improve performance. Additionally, any appropriate thermal or cryogenic modality can be used in conjunction with the above processes.

In addition to inspecting and decontaminating the lumens of medical instruments, it is important to document, track, and report the utilization, care, handling, processing, and findings related to this examination, and decontamination of the medical devices throughout their usable life. To facilitate this, the present invention makes use of a unique device identifier (“UDI”) for each medical device, which allows the device to be tracked throughout its distribution and use.

The UDI assigned to a given device can be determined by reading or scanning a code, which can be a serial number or a barcode, including matrix (or two-dimensional) barcodes, such as a QR code. Alternatively, the UDI can be determined using another optical identifier or an RFID tag (radio-frequency identification). As noted above, in certain embodiments, the imaging portion (160) of the borescope (30), in addition to its multiple inspection functions, is also be used as a scanner for a bar code or other graphical or optical indicia that act as a UDI for the medical device.

At the start of a current device inspection and sterilization as described above (or at some time during or at the conclusion thereof), the UDI is obtained from the medical device. During or at the end of the current processing of the medical device, information concerning the current processing of the device is digitally associated with that UDI, which can be manually entered or automatically uploaded to a local or remote computing device or network, such as server (60). As a result of documenting the current sterilization of the device so that the additional data regarding this current processing of the device is thereafter associated with that device's UDI, a subsequent individual handling the device is able to access and add to this device data.

Using the UDI, the subsequent handler can retrieve the device-specific data for that medical device. This will include not only basic information, such as the manufacturer, serial number, age, and purchase date, but will also include information concerning past sterilization and/or sterility assurance procedures that have been conducted for that particular device.

In certain advantageous embodiments, the additional data relating to the current processing of the device is added to the existing data (retrieved with the UDI) by adding it to a distributed ledger, such as a blockchain.

In this way, handlers of the medical device are able to identify, track, document, archive, and communicate inspection findings, repair history, and sterilization history associated with particular medical devices, as well as their association with other devices, the instrument sets they are located or stored in, the procedures they are used in, the clinicians and medical institutions who have used them, the personnel and institutions who have inspected, cleaned, repaired, stored transported and used them, the environments in which they are used, the areas in which they are stored, and the places in which they are trafficked.

Tracking the specific uses of devices along with the sterilization thereof also enables the sterilization process to be improved in the future. For example, a device used in an orthopedic procedure will typically be soiled with blood and bone, while a device used in a gastrointestinal procedure with be soiled with feces. By tracking these different uses along with the particular decontamination procedures employed, the system is able to track which decontamination measures work best. Thus, with the aid of artificial intelligence, the system can assess which decontamination processes are best suited for specific types of instruments and after specific types of procedures. The system's artificial intelligence engine creates correlative data on findings by instrument, procedure, provider, patient, disease state, pathogens, region, geography, etc. As a result, it determines the best practices for disinfection, cleaning, utilization, etc. based on aggregation of findings and outcomes.

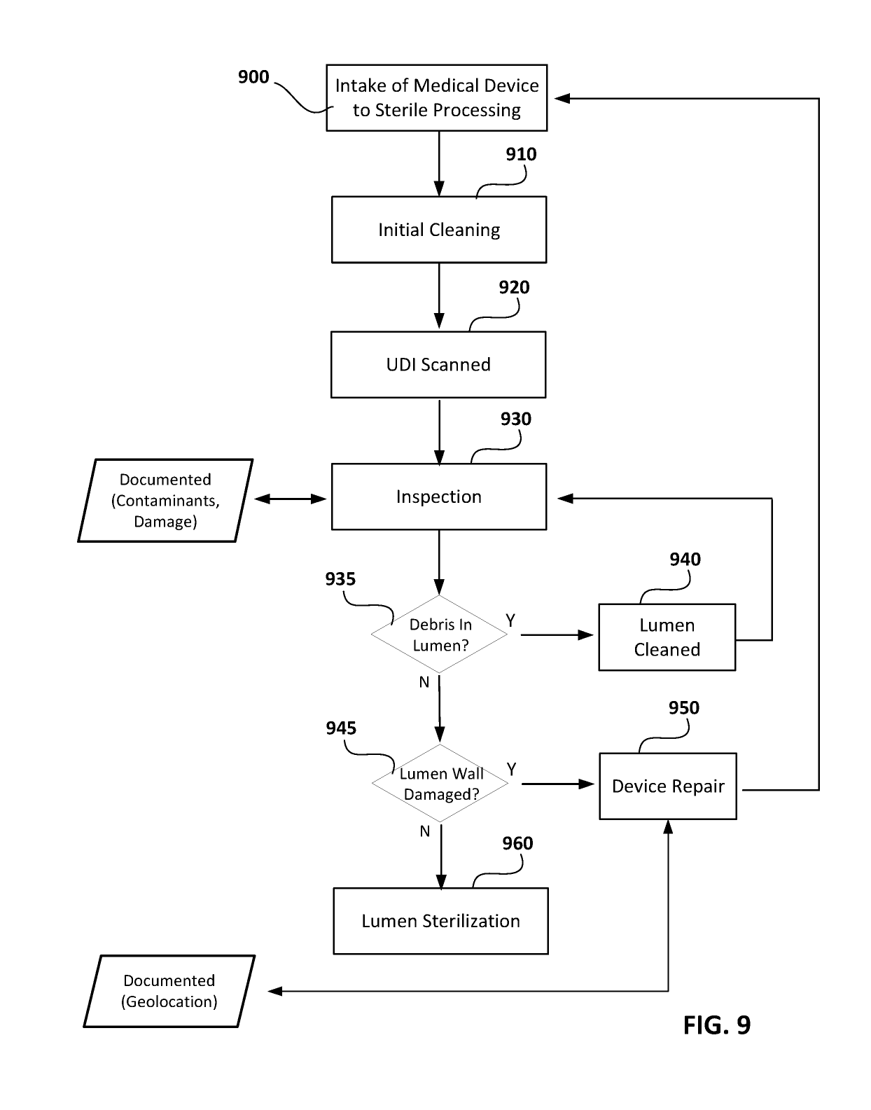

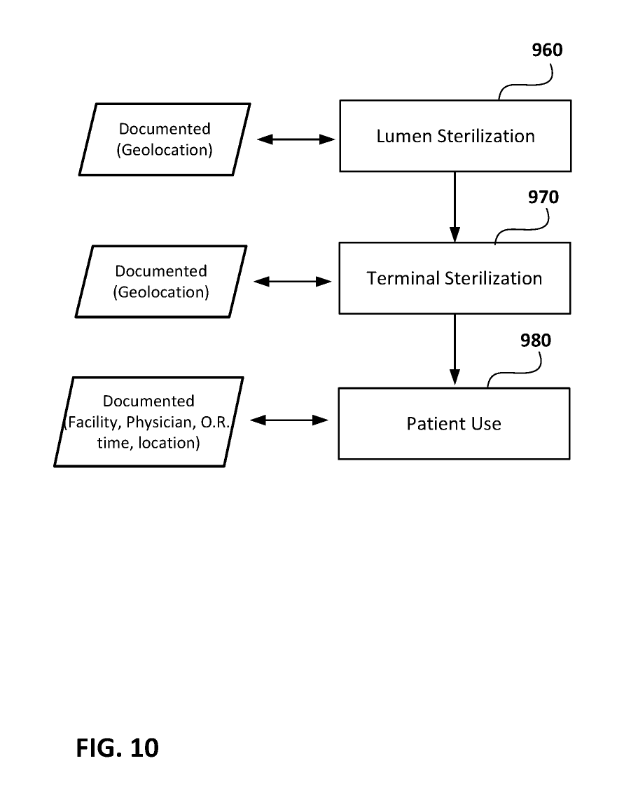

The basic operation of an example of the aforementioned documentation is shown in FIGS. 9-10. Referring first to FIG. 9, a medical device is first received for processing (900). The device receives an initial clean (910), for example, with soap and water. The Unique Device Identifier is then scanned (920), and subsequently inspected (930). Upon inspection, the details of the inspection, including any detected contaminants or damage, are documented in the blockchain.

If the inspection reveals debris in the medical device lumen, the lumen is cleaned (940), for example, with the above described balloon catheter or brush, and is then inspected again. If no debris is observed, the inspection next assesses whether there is any structural damage to the device lumen. If damaged, the device is sent to repair (950), at which time the geolocation of the device is documented in the blockchain. After repaired, the medical device will return to intake for processing.

If the inspection determines the medical device lumen is not damaged, the lumen is then sterilized (960). Turning to FIG. 10, when the device lumen is sterilized (960), the geolocation is again documented in the blockchain. Next, the medical device is terminally sterilized (970), at which point its geolocation is again documented in the blockchain. The sterile device is then wrapped, and subsequently used for a patient (980). At this time, various details relating to the use, such as the medical facility, physician, operating room, type of procedure, date & time, and city, state, country, etc. area all documented in the blockchain.

In this way, users can more efficiently identify individual medical devices that may be possible vectors for patient infection and present a greater risk of device failure.

It should be understood that the foregoing is illustrative and not limiting, and that obvious modifications may be made by those skilled in the art without departing from the spirit of the invention. Although the invention has been described with reference to embodiments herein, those embodiments do not limit the scope of the invention. Accordingly, reference should be made primarily to the accompanying claims, rather than the foregoing specification, to determine the scope of the invention.

What is claimed is:

1. A method of ensuring the structural integrity and decontamination of a medical device, the method comprising:
inserting a distal end of a borescope into a lumen of a medical device, the distal end of the borescope having an imaging portion and at least one decontamination portion;
determining whether the wall of the lumen has been damaged by visually inspecting the wall of the lumen with the imaging portion of the borescope;
determining whether contaminants reside on the wall of the lumen by visually inspecting the wall of the lumen with the imaging portion of the borescope; and
decontaminating the wall of the lumen with the decontaminating portion of the borescope.

2. The method of claim 1, wherein the at least one decontaminating portion comprises an ultraviolet light source, and the step of decontaminating the wall of the lumen comprises sterilizing the wall with ultraviolet light from the ultraviolet light source.

3. The method of claim 2, wherein the at least one decontaminating portion further comprises a fluid delivery device, and the step of decontaminating the wall of the lumen comprises delivering a fluid decontaminant to the wall via the fluid delivery device.

4. The method of claim 3, wherein the step of decontaminating the wall of the lumen comprises sterilizing the wall by delivering a fluid sterilant thereto via the fluid delivery device.

5. The method of claim 3, wherein the step of decontaminating the wall of the lumen comprises delivering a fluid bactericide thereto via the fluid delivery device.

6. The method of claim 1, wherein the at least one decontaminating portion comprises a fluid delivery device, and the step of decontaminating the wall of the lumen comprises delivering a fluid decontaminant to the wall via the fluid delivery device.

7. The method of claim 6, wherein the step of decontaminating the wall of the lumen comprises sterilizing the wall by delivering a fluid sterilant thereto via the fluid delivery device.

8. The method of claim 7, wherein the fluid sterilant comprises peracetic acid.

9. The method of claim 7, wherein the fluid sterilant comprises hypochlorous acid.

10. The method of claim 7, wherein the fluid sterilant comprises ethylene oxide.

11. The method of claim 6, wherein the step of decontaminating the wall of the lumen comprises delivering a fluid bactericide thereto via the fluid delivery device.

12. The method of claim 6, wherein fluid delivery device comprises a nebulizer for delivering an atomized liquid to the wall of the lumen.

13. The method of claim 1, further comprising the steps of:
retrieving device processing data digitally associated with a unique device identifier for the medical device; and
adding to the device processing data additional device processing data resulting from the steps of determining whether the wall of the lumen has been damaged, determining whether contaminants reside on the wall of the lumen, and decontaminating the wall of the lumen.

14. The method of claim 13, wherein the step of adding to the device processing data comprises adding the additional device processing data to a distributed ledger.

15. The method of claim 14, wherein the step of adding the additional device processing data comprises adding the information to a blockchain.

16. The method of claim 13, wherein the step of retrieving device processing data digitally associated with a unique device identifier comprises scanning the unique device identifier with the imaging portion of the borescope.

17. The method of claim 1, further comprising the following steps, after the step of determining whether contaminants reside on the wall of the lumen and before the step of decontaminating the wall of the lumen:
withdrawing the distal end of the borescope from the lumen of the medical device;
inserting a catheter having a balloon with a mesh thereon into the lumen;
cleaning the lumen with the balloon;
withdrawing the catheter from the lumen; and
reinserting the borescope into the lumen.

18. The method of claim 1, further comprising the following steps, after the step of determining whether contaminants reside on the wall of the lumen and before the step of decontaminating the wall of the lumen:
withdrawing the distal end of the borescope from the lumen of the medical device;
inserting a brush into the lumen;
cleaning the lumen with the brush;
withdrawing the brush from the lumen; and
reinserting the borescope into the lumen.

19. The method of claim 1, further comprising the step of sending the medical device for repair if the step of determining whether the wall of the lumen has been damaged determines that the wall has been damaged.

20. The method of claim 1, further comprising the steps of:
capturing an image of the wall of the lumen while visually inspecting the wall with the imaging portion of the borescope; and
storing the image.

21. The method of claim 1, further comprising the steps of:
recording a video of the wall of the lumen while visually inspecting the wall with the imaging portion of the borescope; and
storing the video.

22. The method of claim 1, wherein the imaging portion comprises a CMOS sensor.
